(19) United States
(12) Patent Application Publication
(10) Pub. No.: US 2008/0299234 A1
(43) Pub. Date: Dec. 4, 2008

(54) Medication Comprising Plant Extracts as a Lipase Inhibitor

(76) Inventor: Jurgen Schrezenmeir, Kiel (DE)

Correspondence Address: EDWIN D. SCHINDLER, FIVE HIRSCH AVENUE, P.O. BOX 966, CORAM, NY, 11727-0966, US

(21) Appl. No.: 11/908,598

(22) PCT Filed: Mar. 9, 2006
(86) PCT No.: PCT/DE2006/000428

§ 371 Date: May 2, 2008

(30) Foreign Application Priority Data
Mar. 17, 2005 (DE) 10 2005 012 832.7

(51) Int. Cl. A61K 36/53

(52) U.S. Cl. 424/745

(57) ABSTRACT

A medication having plant extracts is administered to a person as a lipase inhibitor. The medication includes one or more of the following plant extracts: cardamom, marjoram, thyme, chili (Capsicum frutescens), evening primrose, oregano leaves (Oreganum vulgare), camomile leaves (Chamomilla recutica), nutmeg (Myristica fragrans), apple marc (pomace), corn germ, almond, hazelnut, walnut, pumpkin germ, Cuphea wrightii, oil flax (Linum usitatissimum), burdock (Arctium lappa L), flax, germs of Helianthus annuus (sunflower), marigold (Calendula officinalis), Echinops banaticus, Mallotus philippinensis, colza, sesame, black caraway, Cynoglossum officinale, lapacho tea, arctostaphylos uva-ursi, stone flower (Flor de piedra), propolis, Primula veris, Magnolia officinalis and celery (Apium graveolens).

Excessive dietary intake of fat can cause a number of disorders, such as obesity, hyperlipemia and diabetes type II, central symptoms of the metabolic syndrome. Elevated blood triglycerides (TG) and glucose levels after food intake are an independent risk factor for insulin resistance which plays a central role in the development of the metabolic syndrome. One possibility to reduce postprandial triglyceride levels is the inhibition of intestinal lipid hydrolyzing enzymes. The pancreatic lipase (E.C. 3.1.1.3) is essential for degradation and absorption of dietary lipids in the intestinal tract. Thus its inhibition leads to a prevention or delay of the intestinal absorption of food TG and consequently to a decrease of blood TG. Obesity is an important risk factor for type 2 diabetes. It is well established that weight reduction improves glycemic control and leads to a reduced cardiovascular disease risk factor. It is generally regarded as difficult to achieve weight loss and to retain a reduced weight. Tetrahydrolipstatin (THL, Orlistat, Xenical®) is a commercially available lipase inhibitor and obtained from Streptomyces toxytricini. After oral administration Orlistat inhibits lipid degradation by blocking the active site of the pancreatic lipase. In clinical trials Orlistat resulted in weight reduction and in an improvement of postprandial lipid profile (lowering of postprandial triglycerides, LDL-cholesterol, and that total cholesterol improvement of the LDL/HDL ratio.

It was also possible to reduce the occurrence of type II diabetes with Orlistat. The application of Orlistat leads to undesired side effects like oily or fatty stools. A smoother lipase inhibitory action is achieved by the administration of plant extracts or an active agent thereof may simply result in a delay instead of a complete prevention of lipid absorption and thus could prevent oily or fatty stools.

Systematically this can be achieved by means of active ingredients that are themselves resorbed or inactivated during digestion and are thus removed from the reaction weight with the lipase-colipase fat complex.

The administration of lipase inhibitors is regarded as a useful medication for patients with metabolic syndrome symptoms. Additionally, a lipase-inhibiting plant extract or components isolated therefrom can be used as food supplements.

Several patents mention the use of plant extracts as possible inhibitors for lipase in the digestive tract. Specifically, these are the Japanese applications with the following numbers: JP 2003 026 585, JP 2002 275 077, JP 2002 047 194.

Several publications describe the positive effects of plant extracts on the postprandial lipid profile, namely the following substances Cyclocarya paliurus, grape seeds, sage, Cassia mimosoides, L. var. nomame Makino and Alpinia officinarum.

Furthermore, it is the object of the invention to determine other plants, which can be used as lipase inhibitors.

To achieve this object, extracts or essences of the following substances and mixtures thereof are mentioned.

cardamom
marjoram
thyme
chili (Capsicum frutescens)
evening primrose
oregano leaves (Oreganum vulgare)
camomile leaves (Chamomilla recutica)
nutmeg (Myristica fragrans)
apple marc (pomace)
corn germ
almond
hazelnut
walnut
pumpkin germ
Cuphea wrightii (e)
evening primrose
oil flax (Linum usitatissimum)
burdock (Arctium lappa L) (e)
flax (e)
germs of Helianthus annuus (sunflower) (e)
marigold (Calendula officinalis) (e, h)
Echinops banaticus
Mallotus philippinensis (e)
colza (e)
sesame (e)
black caraway (m)
Cynoglossum officinale
lapacho tea (e)
arctostaphylos uva-ursi
stone flower (Flor de piedra)
propolis
Primula veris
Magnolia officinalis
celery (Apium graveolens)

### Description of the Preparation of the Extracts

### a) CO₂ Extracts:

High-pressure extraction with supercritical carbon dioxide. Contains no water, no sugars, no proteins

### Cardamom

Used part of the plant: seed with capsule from Elettaria cardamomum

The crude material obtained from Guatemala
Extract consistency: oily fluid
Extract contains 72% essential oil
Other ingredients:
α-pinene, β-pinene, α-myrcene, 1,8-cineol, linalool, α-terpineol, linalyl acetate, terpinyl acetate

### Marjoram:

Used part of the plant: leaves of Origanum majorana
Extract consistency: oily fluid
Extract contains 80% essential oil
Other ingredients:
sabinene, cis- and trans-sabinene hydrates, terpinen-4-ol,
sabinene hydrate acetate

### Thyme

Used part of the plant: Leaves of Thymus vulgaris
The crude material obtained from Germany
Otherwise see above for cardamom.
Chili (Capsicum frutescens):
Used part of the plant: husk
Crude material obtained from India
Extract standarized with sunflower oil
Extract consistency: otherwise see above

### Evening Primrose:

Used part of the plant: seed
Extract stabilized with tocopherol
Extract consistency: oily liquid

### Oregano Leaves:

Extract consistency: oily fluid
Ingredients: 0.18% terpinene, 2.6% p-cymene, 0.07% limonene, 62% carvacrol, 1.5% caryophyllene, 0.33% thymol, 0.49% 1.8-cineol, 0.17% y-terpinene, 0.2% β-terpinene, 1.5% 4-terpineol, 0.58% α-terpineol, 23.2% thymoquinone,

### Camomile Leaves:

Extract consistency: oily fluid
20-25% bisabolol,
5-20% bisabolol oxides,
1.0-3.0% matricin

### Nutmeg

Crude material obtained from Indonesia
Extract consistency: oily liquid
Ingredients: 19-24% myristicin
1-3% safrol
   b) The following extracts were manufactured:
   10 g of crude material powder was dissolved in
   e) 50 ml pure ethanol at 37° C.

### w) Water at 37° C.

### h) Water at 95° C.

m) 60% ethanol at 37° C.
and incubated in a shaking water bath for 2 hours. Afterwards the extract was stirred on a magnet stirrer for 30 minutes and centrifuged at 6000 rpm for 10 minutes. Subsequently the solvent was removed in a Speed Vac Concentrator.

### Apple Marc (Pomace) (e):

extract Consistency: viscous
liquid

### Corn Germs (e):

Extract consistency: viscous liquid

### Almond (e):

Extract consistency: viscous liquid

### Hazelnut (e):

Extract consistency: viscous liquid

### Walnut, Sort S (e):Extract Consistency:

viscous liquid

### Pumpkin Germ (e):

Extract consistency: viscous liquid

Cuphea wrightii (e):
Extract consistency: viscous liquid

### Evening Primrose (e):

Used part of the plant: seed
Extract consistency: viscous liquid

### Oil Flax (165) (e)

Extract consistency: viscous liquid

### Oil Flax (162) (e)

Extract consistency: viscous liquid

### Burdock (e):

Used part of the plant: root
Extract consistency: viscous liquid

### Flax (e)

Extract consistency: viscous liquid
Germs of Helianthus annuus (Sunflower) (e): Extract
consistency: viscous liquid
Marigold (Calendula officinalis) (e, h)
Extract consistency: viscous liquid
Echinops banaticus (154:e. 152:e):
Extract consistency: viscous liquid
Mallotus philippinensis (e):
Extract consistency: viscous liquid

### Colza (e):

Extract consistency: viscous liquid

### Sesame (e):

Extract consistency: viscous liquid

### Black Caraway (m):

Cvnoglossum officinale (157.e):

Lapacho Tea (e):

Used part of the plant: Inner bark of the taheebo tree (Tabebuia impetiginosa)

Extract consistency: Viscous liquid

### c) Other Extracts:

Arctostaphylos uva-ursi

Used part of the plant: Leaves of the arctostaphylos uva-ursi

Extraction solvent: water

Extract consistency: powder

Ingredient: Arbutin

Stone Flower (Flor de piedra):
Used part of the plant: Extraction
solvent: 69% ethanol (v/v)
Extract consistency: Dry residue: 1.0%

### Propolis:

Propolis is a mixed product comprising material collected by bees from pollens and tree barks, and a secretion of bees themselves

Extraction solvent: propylene glycol
Extract consistency: liquid
Primula veris:

Used part: Leaves and roots of Primula veris

Extraction solvent: 50% ethanol (v/v)

Extract consistency: powder

Magnolia officinalis:
Used part of the plant: bark
Extraction solvent: alcohol and water
Extract consistency: powder
Celery (Apium graveolens)
Used part of the plant: tuber
Extraction solvent: unknown
Extract consistency: oily liquid
Ingredient: celery oil

The lipase inhibiting substance in the plant extracts named in the patent publication have not been yet identified:

### Description of Test Solution Preparation and of the Methods for Enzyme Activities Determination:

### Preparation of the Test Solution

To prepare the test solution of extract powders and viscous liquids, 200 mg of the extract material were dissolved in 1 ml of the appropriate solvent by intensive stirring. After a centrifugation of 3400 rpm for 5 minutes the supernatant was taken as test solution. To match the ratio between the concentration of solid extract and enzyme molarity in the final assay, the test solution was diluted 1:200 for determination of the alkaline phosphatase.

To prepare the test solutions of CO₂ extracts and oleoresins 100 uL of oily fluid extract were dissolved in 900 μL of the appropriate enzyme buffer (see above) and were treated with ultrasonic with 100 watts for 5 minutes.

### Pancreatic Lipase

Two lipase determination assays were conducted in order to detect the activity of the pancreatic lipase in vitro:

1. The assay for the determination of the lipase activity consists of:

200 μL of a 50 mM/L BICIN buffer (pH 8.0) containing 1% technical gum arabic,

50 μL plant extract test solution, and

25 μL of a 0.35 mM/L pancreatic lipase solution

This mixture was incubated for 20 minutes at 37° C.

The final measurement of pancreatic lipase activity was conducted in an autoanalyser for clinical chemistry (Konelab, Kone), using a lipase colorimetric test with a diacylglycerol with methylresorufin in third position as substrate and colipase and bile salts as essential cofactors. This method is specific for the pancreatic lipase and is based upon the cleavage of methylresorufin from the substrate by the enzyme.

The pipette procedure in the autoanalyser in order to detect pancreatic lipase activity was conducted as follows:

1. 60 μl reagent 1: colipase and cholate

2. 2 μl test solution+10 μl wash solution

3. 180 seconds' incubation

4. 40 μl reagent 2: Colorimetric substrate and cholate

5. 120 seconds' incubation

Lipase activity was determined from 12 measuring points after 83 seconds. Methylresorufin was measured photometrically at 75 nm.

2. Lipase inhibition under influence of a plant extract was confirmed with a second lipase activity method measuring the release of free fatty acids during hydrolysis of triolein by the enzyme.

To prepare the incubation mix a ready-to-use solution (Reagent 1, R1) is used. R1 consists of:

26 mmol/L tris buffer, pH 9.2
19 mmol/L sodium desoxycholate
0.1 mmol/L calcium chloride
0.3 mmol/L triolein
300 KU/L colipase

The incubation mix has the following composition:

50 μL 26 mmol/L tris buffer, pH 9.2
50 μL 9.7 mmol/L triolein emulsion in R1
50 uL 0.134 mol/L pancreatic lipase in 26 mmol/L tris buffer pH 9.2
50 μL test solution

1% methyl cellulose in 26 mmol/L tris buffer (pH 9.2) was used for investigating CO₂ extracts.

After 30 minutes incubation at 37° C. in a water bath the reaction was stopped by addition of 3 ml of a chloroform/heptane/methanol mixture (50:49:1). The released free fatty acids were extracted from the reaction mix by intensive stirring for 10 minutes. After centrifugation (3,400 rpm, 10 minutes) a 2 ml aliquot was taken from the chloroform phase and 1 ml of a copper reagent (94 ml pure water, 6 ml 1 N NaOH, 6.45 mmol/L Cu(NO₃)₂, 0.1 mol/L triethanolamine, 33% (w/v) NaCl) was added. This mixture was stirred intensively for 10 minutes and centrifuged for 10 min at 3400 rpm 1 ml was taken from the supernatant liquid and 1 ml of chloroform with 0.1% (w/v) bathocuproine and 0.05% (w/v) 3-tert-butyl-4-hydroxyanisol were added. The fatty acid-copper-bathocuproin-complex was finally photometrically determined at 480 nm in a standard photometer.

To ensure that the decrease in lipase activity is not due to an unspecific enzyme inhibition or denaturing of the enzyme complex, the activities of α-amylase and alkaline phosphatase were determined in addition. An inhibition of the α-amylase could result in a lowering of postprandial blood glucose levels and is regarded as additional positive effect for patients with diabetes.

### α-Amylase

The amylase activity measurement under influence of plant extracts were conducted as follows:

The incubation mix for the determination of the amylase activity consists of a total of 250 μL:

200 μL 50 mM/L MOPSO buffer (pH 8.0) containing 1% gum arabic

25 μL plant extract solution, and

25 μL of a 0.35 mM/L α-amylase solution

This mixture was incubated for 20 minutes at 38° C. The final measurement of pancreatic lipase activity was conducted in an autoanalyser for clinical chemistry (Konelab, Kone).

### Principle of the Chemical Reaction:

1. ethylene-p-nitrophenol-(glucose)₇→ethylidene-(glucose)₃₋₄+p-nitrophenol-(glucose)₂₋₄
2. p-nitrophenol-(glucose) residues→p-nitrophenol (A=405 nm)+glucose

The pipette procedure in the autoanalyser in order to detect α-amylase activity was conducted as follows:

1. 120 μl reagent: substrate+α-glucosidase
2. 300 seconds incubation
3. 3 μL test solution
4. 180 seconds' incubation

α-amylase activity is determined by measuring 5 points in 120 seconds.

### Alkaline Phosphatase

The alkaline phosphatase activity measurement under influence of plant extracts was conducted as follows:

The incubation mix for the determination of the lipase activity consisted of:

200 μL of a 50 mM/L HEDTA buffer (pH 8.0) containing,

25 μL plant extract test solution, and

25 μL of a 0.014 mM/L alkaline phosphatase solution

This mixture was incubated for 20 minutes at 22° C. The final measurement of pancreatic lipase activity was conducted in an autoanalyzer for clinical chemistry (Konelab, Kone).

### Reaction Equation

2-amino-2-methyl-1-propanol+4-nitrophenylphosphate->(2-amino-2-methyl-1-propanol) phosphate+4-nitrophenoxide (λ=409 nm)

The pipette procedure in the autoanalyser in order to detect alkaline phosphatase activity was conducted as follows:

1. 150 μl reagent
2. 300 seconds' incubation
3. 3 μL test solution
4. 120 seconds' incubation

The activity of the alkaline phosphatase was determined by means of 5 measurement points in 22 seconds.

### Verification by Animal Experiment

For further examination and verification of the results determined in vitro, the triglyceride-reducing effect was tested in an animal experiment.

To this end a lipid stress test was carried out in rats with Triton WR-1339 to inhibit the triglyceride elimination from the blood and the postprandial rise in plasma triglyceride values over four hours The following test substances were used:

### Proteins and Peptides

Protamine: strongly basic core protein (from 32 AS 21×arg), obtained from fish sperm, triglyceride reducing (Tsujita et al, 1996)
Peptide (D4 Globin Digest): enzymatic hydrolyzate of bovine erythrocyte globin, triglyceride-reducing (Kagawa et al., 1996, 1998)
VVYP: fragment 32-35 of the β-chain of bovine haemoglobin, triglyceride-reducing (Kagawa et al., 1996, 1998)
LVYP: fragment 73-76 of bovine β-casein

### Extracts

Ethanolic extracts
   Tabebuia impetiginosa (Lapacho) leaves
   Arctium lappa L (burdock)
   Seeds of Linum usitatissimum (linseed)
   Seeds of helianthus annuus (sunflower)
   Calendula officinalis (marigold)
Oleoresin
   Seeds of Carum carvi (caraway)
   CO₂ Extracts
   Fruit with husk of Elettaria cardamomum (cardamom)
   Leaves of Thymus vulgaris (thyme)
Propolis

### Extracts

Ethanolic extracts
   Tabebuia impetiginosa (Lapacho) leaves
   Arctium lappa L (burdock)
   Seeds of Linum usitatissimum (linseed)
   Seeds of helianthus annuus (sunflower)
   Calendula officinalis (marigold)
Oleoresin
   Seeds of Carum carvi (caraway)
CO₂ Extracts
   Fruit with husk of Elettaria cardamomum (cardamom)
   Leaves of Thymus vulgaris (thyme)
Propolis

Orlistat as positive control.

As result, the rise in postprandial triglyceride values in the plasma of rats after administration of the test substances was recorded. This shows that no triglyceride-reducing effect was found for either protamine, peptide D4 or the tetrapeptide. Likewise for some extracts in this test, no desired effect could be ascertained. They include linseed, sunflower, marigold and propolis.

With other extracts, however, a partly very clear reduction of the triglycerides could be observed. They include caraway with a reduction of about 3%±9%, thyme with a reduction of 7% ±7%, burdock with a reduction of 12%±6%, cardamom with a reduction of 13%±7% and in particular lapacho tea extract, with a significant reduction of 16%±7%. In the experiment, no differentiation was made between which ingredients of the extracts impart the effect observed overall. This leaves scope for performing further tests in animals, and gives an indication of the need for human studies.

In general, with this animal experiment, it was possible to demonstrate that, of the active substances characterised as effective according to the invention, some actually showed the same effect in an animal experiment.

Likewise it became clear that this effect is by no means linearly transferable, and in individual cases is even overlaid by other influences, which in an extreme case may lead to an inversion of the effect. From the experiment, however, it can be deduced that the substances listed here as worthy of protection in the sense of a specific lipase reduction deserve further examination, and that very concrete results can be expected. Further details and features of the invention are explained below in greater detail with reference to examples. However, they are not intended to limit the invention but only explain it. In schematic view,

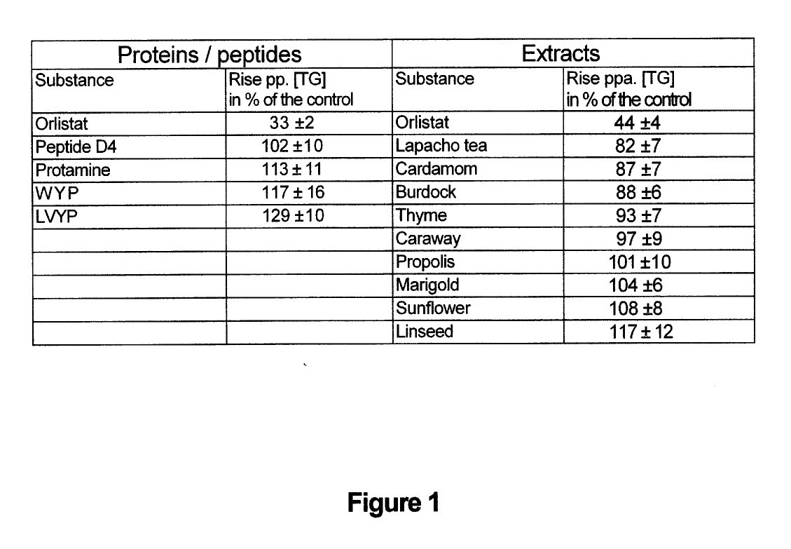

FIG. 1 shows the rise in postprandial triglyceride values in the plasma of rats after administration of the test substances, and

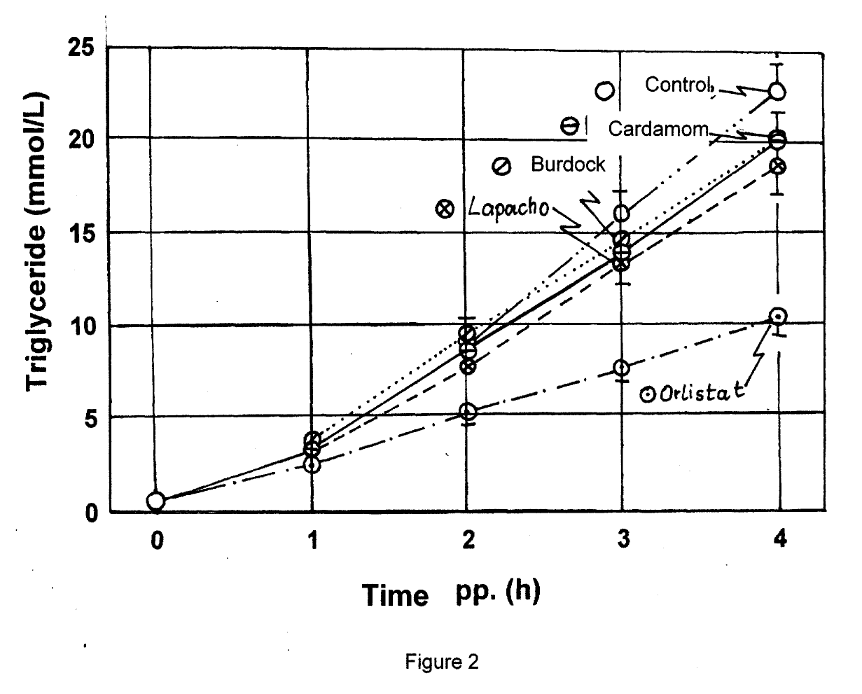

FIG. 2 shows the posprandial triglyceride values in rat plasma after a lipid stress test.

In detail, the figures show:

FIG. 1 shows in tabular form the effect on the triglyceride value of the substances administered to the rats about 0.05 hours after food intake. This distinguishes between the proteins and peptides and the extracts of natural ingredients. It is significant that the proteins and peptides in this test configuration do not lead to the desired reduction but in some cases to a significant increase of the triglyceride values.

That also applies to some natural materials. Other natural substances are however effective in the desired direction. Particularly significant here is the effect of lapacho tea from the inner bark of the taheebo tree (Tabebuia impetiginosa).

FIG. 2 shows the curves for triglyceride values as a function of the time after food intake. As expected, the triglyceride values with the administration of Orlistat are the lowest. For the positively acting extracts already listed in FIG. 1, however, it was also possible to achieve a reduction of the triglycerides as desired in this experimental period of four hours. Those that have proved effective are burdock, lapacho tea and cardamom. The comparatively particularly low rise in triglyceride values on administration of Orlistat correspond to expectations and indicate that the animal experiment is relevant to the invention within the specification carried out.

1-2. (canceled)

3. A method for administering a medication that acts as a lipase inhibitor to a person, comprising the steps of:
formulating a medication that acts as a lipase inhibitor, said medication comprising a plant extract component selected from the group consisting of cardamom, marjoram, thyme, chili (Capsicum frutescens), evening primrose, oregano leaves (Oreganum vulgare), camomile leaves (Chamomilla recutica), nutmeg (Myristica fragrans), apple marc (pomace), corn germ, almond, hazelnut, walnut, pumpkin germ, Cuphea wrightii, oil flax (Linum usitatissimum), burdock (Arctium lappa L), flax, germs of Helianthus annuus (sunflower), marigold (Calendula officinalis), Echinops banaticus, Mallotus philippinensis, colza, sesame, black caraway, Cynoglossum officinale, lapacho tea, arctostaphylos uva-ursi, stone flower (Flor de piedra), propolis, Primula veris, Magnolia officinalis, celery (Apium graveolens) and a combination thereof; and,
orally administering said medication to a person.
